(19) United States
(12) Patent Application Publication
(10) Pub. No.: US 2023/0014312 A1
(43) Pub. Date: Jan. 19, 2023

(54) TREATMENT APPARATUS AND TREATMENT METHOD

(71) Applicant: TERUMO KABUSHIKI KAISHA, Tokyo (JP)

(72) Inventors: Satoru SUEHARA, Ashigarakami-gun (JP); Mayu HATA, Yokohama-shi (JP)

(73) Assignee: TERUMO KABUSHIKI KAISHA, Tokyo (JP)

(21) Appl. No.: 17/952,742

(22) Filed: Sep. 26, 2022

Related U.S. Application Data
(63) Continuation of application No. PCT/JP2021/009430, filed on Mar. 10, 2021.

(30) Foreign Application Priority Data
Mar. 30, 2020 (JP) 2020-060401

(51) Int. Cl. A61N 5/06

(52) U.S. Cl. CPC A61N 5/062; A61N 5/0603; A61N 2005/0611; A61N 2005/0659

(57) ABSTRACT

Provided are a treatment apparatus and a treatment method capable of effectively treating cancer tumor cell (for example cancer in a range including at least a part of a cervix). A treatment apparatus is configured to irradiate an antibody-photosensitive substance bound to a tumor cell with excitation light, and includes: a main shaft including a distal portion and a proximal portion; a distal structure portion disposed on a distal side of the main shaft and formed to be larger than the main shaft in a radial direction of the main shaft; a distal shaft protruding from the distal structure portion toward the distal side; and at least one irradiation unit configured to emit the excitation light of the antibody-photosensitive substance from the distal shaft and the distal structure portion.

### CROSS-REFERENCES TO RELATED APPLICATIONS

This application is a continuation of International Application No. PCT/JP2021/009430 filed on Mar. 10, 2021, which claims priority to Japanese Patent Application No. 2020-060401 filed on Mar. 30, 2022, the entire content of both of which is incorporated herein by reference.

### TECHNOLOGICAL FIELD

The present invention generally relates to a treatment apparatus and a treatment method for cervical cancer.

### BACKGROUND DISCUSSION

The number of patients with cervical cancer has an increasing tendency, and in particular, the number of young female patients in their 20s and 30s is increasing. In current treatment for cervical cancer, standard treatment is to remove an entire uterus from an early stage (stage I), but for young patients, local treatment is required to conserve the uterus in order to maintain fertility. Further, in an advanced stage (stage III and subsequent stages), cancer has spread to surrounding tissues and is difficult to remove by surgery, and thus standard treatment is to combine radiation therapy and chemotherapy. However, a five-year survival rate is as low as 50% in stage III and 20% in stage IV, and more effective treatment is required. As the local treatment for cancer, a treatment method using a photoreactive substance is known (for example, see United States Patent Application Publication No. 2018/0113246). In particular, a treatment method using an antibody-photosensitive substance (hydrophilic phthalocyanine) can specifically destroy target cells without destroying non-target cells such as normal cells by irradiating the antibody-photosensitive substance accumulated in a tumor with excitation light (for example, near-infrared rays), and is expected to achieve a high treatment effect while minimizing side effects.

To achieve a high treatment effect by the antibody-photosensitive substance, the antibody-photosensitive substance accumulated in the tumor is required to be reliably irradiated with the near-infrared rays. However, since light is rapidly attenuated due to an influence of a biological tissue, the near-infrared rays have a small penetration depth, and it is extremely difficult to non-invasively irradiate a solid cancer with light having energy required for treatment from a body surface. Therefore, a method for reliably irradiating the tumor in a body with light while reducing invasiveness as much as possible is required. In the case of cervical cancer, cancer often spreads over a wide range of a cervical canal, and a method for irradiating cancer in a wide range with light from as close as possible is required.

### SUMMARY

The treatment apparatus and treatment method disclosed here effectively treat a tumor cell.

The treatment apparatus is configured to irradiate an antibody-photosensitive substance bound to a tumor cell with excitation light. The treatment apparatus includes: a main shaft including a distal portion and a proximal portion; a distal structure portion disposed at the distal portion of the main shaft, the distal structure portion possessing an outer configuration that is larger in a radial direction of the main shaft than the main shaft in the radial direction of the main shaft; a distal shaft protruding in a distal direction from the distal structure portion so that a distal end of the distal shaft protrudes distally beyond the distal structure portion; and at least one irradiation unit configured to emit, from the distal shaft and the distal structure portion, the excitation light to irradiate the antibody-photosensitive substance.

According to the treatment apparatus described above, the excitation light can be effectively emitted to the antibody-photosensitive substance bound to the tumor cell. By way of example, the excitation light can be effectively emitted to the antibody-photosensitive substance bound to the tumor cell in a state in which the distal shaft is inserted into a cervical canal and the distal structure portion is inserted into a vagina. Therefore, this treatment apparatus can improve a treatment effect of cancer in a range including at least a part of the cervix.

The treatment apparatus may be formed with an irradiation lumen communicating with an inside of a through hole and an inside of the distal shaft, and configured to movably accommodate the irradiation unit, the through hole penetrating from a distal side to a proximal side of the distal structure portion. Accordingly, even if only one irradiation unit is provided, the excitation light can be emitted from the distal shaft and the distal structure portion, and thus a configuration of the treatment apparatus can be simplified and operability can be improved. By moving the irradiation unit, a position where the excitation light is emitted can be appropriately adjusted, and thus the treatment effect can be improved.

The distal structure portion may have a cup shape with a recessed portion formed on the distal side thereof. Accordingly, the excitation light can be effectively emitted to the antibody-photosensitive substance bound to the tumor cell in a wide range including the cervix in a state in which the distal shaft is inserted into the cervical canal and a site or portion surrounding the recessed portion of the distal structure portion is inserted into the vicinity of a vaginal vault. Therefore, this treatment apparatus can improve the treatment effect of cancer in a wide range including the cervix.

The distal structure portion may include a wall portion surrounding the recessed portion and protruding toward the distal side, and the wall portion may include, at a part in a peripheral direction surrounding the recessed portion, a protruding portion having a protruding amount in a distal direction larger than those of other sites. Accordingly, the wall portion can be brought close to the vaginal vault. Therefore, the excitation light can be effectively emitted to the vicinity of the vaginal vault, which is difficult for light to reach, and the treatment effect can be improved.

The distal shaft may be configured to emit the excitation light in a direction substantially perpendicular to an axial center of the distal shaft, and the distal structure portion may be configured to emit the excitation light in a substantially distal direction. Accordingly, the excitation light can be emitted to the tumor cell of the cervix from both the distal shaft and the distal structure portion, and thus the treatment effect can be improved.

The treatment apparatus may further include a detection unit configured to detect fluorescence emitted by the antibody-photosensitive substance. Accordingly, a degree of destruction of the tumor cell due to emission of the excitation light can be checked by a change in the fluorescence detected by the detection unit.

The distal structure portion may be configured to move relative to the main shaft in an axial center direction of the main shaft. Accordingly, the distal shaft can be inserted into the cervical canal in a state in which the distal structure portion is retracted toward a proximal side with respect to the main shaft to secure a visual field. In a state in which the distal shaft is maintained at an appropriate position of the cervical canal, the distal structure portion can be moved and disposed at an appropriate position. Therefore, both the distal shaft and the distal structure portion can be accurately and easily disposed at appropriate positions of the cervical canal and the vagina. Therefore, the excitation light can be emitted from the distal shaft and the distal structure portion to desired positions, and thus the treatment effect can be improved.

The treatment method includes: intravenously administering an antibody-photosensitive substance; inserting a treatment apparatus into a living body 12 to 36 hours after the intravenous administration of the antibody-photosensitive substance, the treatment apparatus including a distal structure portion disposed on a distal portion of an elongated main shaft and a distal shaft protruding in a distal direction beyond the distal structure portion, and the treatment apparatus being configured to emit excitation light of the antibody-photosensitive substance; inserting the distal shaft into a body lumen; inserting the distal structure portion into the living body; emitting the excitation light from the distal shaft to a surrounding tissue to excite the antibody-photosensitive substance; and emitting the excitation light from the distal structure portion to a surrounding tissue to excite the antibody-photosensitive substance.

According to the treatment method described above, the distal shaft can be inserted from, for example, an external uterine ostium into the cervical canal, and the distal structure portion can be inserted into, for example, the vagina, and thus by emitting the excitation light of the antibody-photosensitive substance from the distal shaft and the distal structure portion, the excitation light can be effectively emitted to the antibody-photosensitive substance bound to the tumor cell in a wide range including the cervix. Therefore, this treatment method can improve the treatment effect of cancer in a range including at least a part of the cervix.

The distal structure portion may have a cup shape with a recessed portion formed on a distal side thereof, and in the step of inserting the distal structure portion into the vagina, at least a part of the distal structure portion may be inserted into a vaginal vault. Accordingly, the excitation light can be effectively emitted to the antibody-photosensitive substance bound to the tumor cell in a wide range including the cervix. Therefore, this treatment method can improve the treatment effect of cancer in a wide range including the cervix.

In the emitting of the excitation light from the distal shaft, an irradiation unit configured to emit the excitation light may be disposed inside the distal shaft to emit the excitation light from the irradiation unit, in the emitting of the excitation light from the distal structure portion, the irradiation unit may be disposed inside the distal structure portion to emit the excitation light from the irradiation unit, and the irradiation unit may be moved between the distal shaft and the distal structure portion between the step of emitting the excitation light from the distal shaft and the emitting of the excitation light from the distal structure portion. Accordingly, even if only one irradiation unit is provided, the excitation light can be emitted from the distal shaft and the distal structure portion, and thus the configuration of the treatment apparatus can be simplified and the operability can be improved. By moving the irradiation unit, a position where the excitation light is emitted can be appropriately adjusted, and thus the treatment effect can be improved. An order of emitting the excitation light is not limited. Therefore, the excitation light may be emitted from the distal shaft first, or the excitation light may be emitted from the distal structure portion first. The number of the irradiation unit is not limited to one.

The emitting of the excitation light from the distal shaft and the emitting of the excitation light from the distal structure portion may be performed simultaneously. Accordingly, this treatment method can simultaneously emit the excitation light from various positions and directions, and thus the treatment effect can be improved, and treatment can be efficiently performed in a short time.

The treatment method may further include detecting fluorescence emitted by the antibody-photosensitive substance and checking an intensity of the fluorescence. Accordingly, in this treatment method, the degree of the destruction of the tumor cell due to the emission of the excitation light can be checked by detecting the fluorescence.

The checking the intensity of the fluorescence may be performed in parallel with the emitting of the excitation light. Accordingly, in this treatment method, a tumor can be treated while detecting the fluorescence to check the degree of the destruction of the tumor cell due to the emission of the excitation light, and the treatment effect can be improved.

The step of checking the intensity of the fluorescence may be performed after the emitting of the excitation light. Accordingly, in this treatment method, a result of the destruction of the tumor cell due to the emission of the excitation light can be accurately checked by detecting the fluorescence.

According to a further aspect, a treatment apparatus configured to irradiate an antibody-photosensitive substance bound to a tumor cell with excitation light comprises: a main shaft including a lumen extending throughout the main shaft, the main shaft including a distal portion; a distal structure portion disposed at the distal portion of the main shaft and movable together with the main shaft; an irradiation shaft positioned in the lumen in the main shaft and axially movable relative to the main shaft, the irradiation shaft including an irradiation lumen; the irradiation shaft including a distal portion at which is located a distal shaft, the irradiation lumen extending into a distal portion of the distal shaft; the distal portion of the distal shaft being located distally beyond a distal-most part of the distal structure portion, the irradiation lumen extending through the distal structure portion; the distal structure portion being configured so that an outermost surface of the distal structure portion is radially outwardly of an outer surface of the main shaft and radially outwardly of an outer surface of the distal portion of the distal shaft; an irradiation unit positioned in the irradiation lumen, axially movable in the irradiation lumen and configured to emit, from both the distal structure portion and the distal portion of the distal shaft that is located distally beyond the distal-most part of the distal structure portion the excitation light to irradiate the antibody-photosensitive substance.

### BRIEF DESCRIPTION OF THE DRAWINGS

FIG. 1 is a plan view illustrating a treatment apparatus according to an embodiment.

FIGS. 2A and 2B are schematic views illustrating a vagina and a uterus, in which FIG. 2A illustrates a state of a patient viewed from front, and FIG. 2B illustrates a state of the patient viewed from a left side.

FIG. 3 is a cross-sectional view illustrating a distal portion of the treatment apparatus according to the embodiment.

FIGS. 4A and 4B are plan views illustrating distal shafts according to modifications, in which FIG. 4A illustrates a first modification, and FIG. 4B illustrates a second modification.

FIG. 5 is a plan view illustrating a third modification.

FIGS. 6A to 6E are cross-sectional views illustrating distal structure portions according to modifications, in which FIG. 6A illustrates a fourth modification, FIG. 6B illustrates a fifth modification, FIG. 6C illustrates a sixth modification, FIG. 6D illustrates a seventh modification, and FIG. 6E illustrates an eighth modification.

FIGS. 7A to 7D are cross-sectional views illustrating distal structure portions according to modifications, in which FIG. 7A illustrates a ninth modification, FIG. 7B illustrates a 10th modification, FIG. 7C illustrates an 11th modification, and FIG. 7D illustrates a 12th modification.

FIGS. 8A to 8E are plan views illustrating distal structure portions according to modifications, in which FIG. 8A illustrates a 13th modification, FIG. 8B illustrates a 14th modification, FIG. 8C illustrates a 15th modification, FIG. 8D illustrates a 16th modification, and FIG. 8E illustrates a 17th modification.

FIGS. 9A to 9D are cross-sectional views illustrating distal structure portions according to modifications, in which FIG. 9A illustrates an 18th modification, FIG. 9B illustrates a 19th modification, FIG. 9C illustrates a 20th modification, and FIG. 9D illustrates a 21st modification.

FIGS. 10A to 10C are plan views illustrating irradiation units according to modifications, in which FIG. 10A illustrates the present embodiment, FIG. 10B illustrates a 22nd modification, and FIG. 10C illustrates a 23rd modification.

FIG. 11 is a schematic view illustrating a state in which the distal shaft of the treatment apparatus according to the embodiment is inserted into a cervical canal.

FIG. 12 is a schematic view illustrating a state in which near-infrared rays are emitted from the distal shaft inserted into the cervical canal to tumor cells.

FIG. 13 is a schematic view illustrating a state in which the near-infrared rays are emitted from the distal structure portion in the vagina to the tumor cells.

FIG. 14 is a plan view illustrating a treatment apparatus according to a 24th modification.

### DETAILED DESCRIPTION

Set forth below with reference to the accompanying drawings is a detailed description of embodiments of a treatment apparatus and a treatment method representing examples of the inventive treatment apparatus and treatment method disclosed here. The dimensions or scales on the drawings may be exaggerated or different from actuality/reality for convenience of description and illustration. In the present specification and the drawings, components having substantially the same functional configuration are designated by the same reference numerals, and a detailed description of such components will not be repeated. In the present specification, a side of a device to be inserted into a body lumen is referred to as a “distal side”, and a side to be operated is referred to as a “proximal side”.

A treatment apparatus 10 according to the present embodiment is used for a treatment method for cervical cancer. The treatment apparatus 10 and the treatment method can also be used to simultaneously treat both cervical cancer and vaginal cancer. The treatment method is used for photoimmunotherapy in which an antibody-photosensitive substance bound to cell membranes of target cells is irradiated with near-infrared rays, which serve as excitation light of the antibody-photosensitive substance, to destroy the target cells. The target cells are tumor cells such as cancer cells. In this treatment method, the antibody-photosensitive substance, which is obtained by binding an antibody that specifically binds to only a specific antigen on surfaces of the tumor cells and a photosensitive substance paired with the antibody, is used as a drug. The antibody is not particularly limited, and may be, for example, panitumbab, trastuzumab, HuJ591, pertuzumab, lapatinib, palbociclib, and olaparib. The photosensitive substance is, for example, hydrophilic phthalocyanine which is a substance that reacts with near-infrared rays having a wavelength of about 700 nm (IR700), but is not limited thereto. When IR700 receives near-infrared rays having a wavelength of about 660 nm to 740 nm, a ligand of a functional group that secures water solubility is broken, causing a structural change from water-soluble to hydrophobic. Due to this structural change, membrane protein is extracted, holes are opened in the cell membranes, and water enters the cells, so that the cancer cells can be ruptured and destroyed. IR700 is excited by receiving the near-infrared rays, and emits fluorescence having a wavelength different from an excitation wavelength. For example, IR700 emits fluorescence having a wavelength of 704 nm when excited by receiving near-infrared rays having a wavelength of 689 nm. IR700 changes a structure thereof while emitting the fluorescence by a photoreaction, and stops emitting the fluorescence when IR700 destroyed the tumor cells and finished the role as a drug.

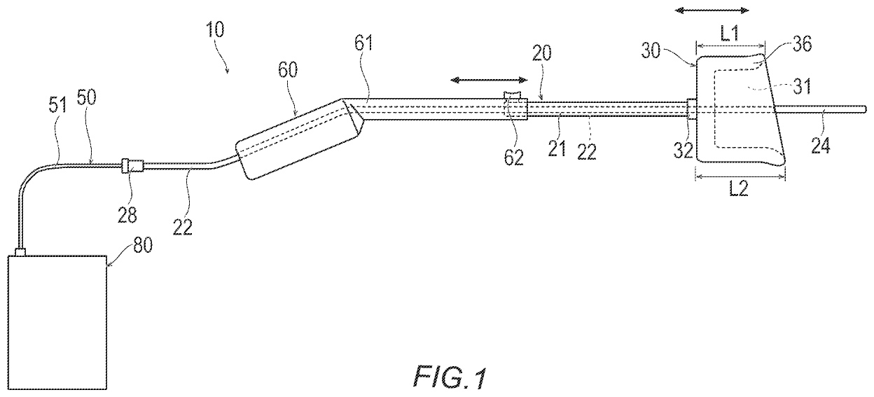

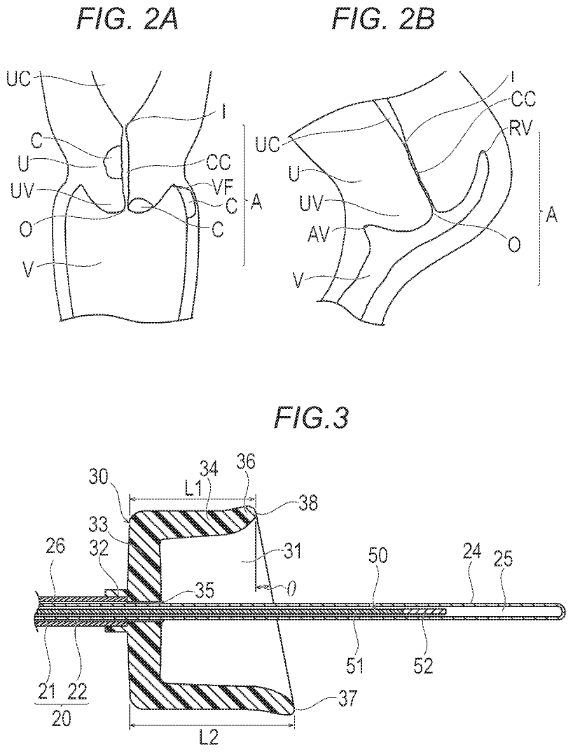

The treatment apparatus 10 illustrated in FIG. 1 can treat, with one device, cervical cancer and vaginal cancer in a wide range A, which is illustrated in FIGS. 2A, 2B, and 11 to 13, and includes a cervix U, an external uterine ostium O, a uterine vagina UV around the external uterine ostium O, a vaginal vault VF, and a site that is near the vaginal vault VF on a vaginal introitus side relative to the vaginal vault VF of a vagina V. The treatment apparatus 10 can emit the excitation light to the antibody-photosensitive substance bound to tumor cells C in a wide range from the cervix U to the vagina V.

A uterus is positioned behind the vagina V, an upper portion of the uterus is connected to left and right fallopian tubes, and the external uterine ostium O at a lower portion of the uterus is connected to the vagina V. The uterus is roughly divided into a uterine corpus and the cervix U, and the cervix U includes a cervical canal CC connected to the external uterine ostium O. The vagina V includes the vaginal vault VF that expands around the external uterine ostium O. The vaginal vault VF is deeper at a posterior vaginal vault RV positioned in a posterior part of the vagina V than at an anterior vaginal vault AV positioned at an anterior part of the vagina V.

First, the treatment apparatus 10 according to the present embodiment will be described.

As illustrated in FIGS. 1 and 3, the treatment apparatus 10 includes an elongated shaft portion 20 including a distal portion and a proximal portion, a distal structure portion 30 provided at the distal portion of the shaft portion 20, an operation portion 60 connected to the proximal portion of the shaft portion 20, and an elongated irradiation unit 50 that emits light. The treatment apparatus 10 is used by being connected to a light output device 80.

The shaft portion 20 includes a main shaft 21 which is a tubular body extending from the operation portion 60 in a distal direction, and an irradiation shaft 22 that accommodates the irradiation unit 50.

The main shaft 21 is a tubular body that supports the distal structure portion 30. The main shaft 21 accommodates the irradiation shaft 22 in a lumen thereof. The main shaft 21 is a circular tube extending linearly, but may be bent or may not be a circular tube. A proximal portion of the main shaft 21 is slidable with respect to a casing 61 of the operation portion 60 and is fixed to a movement operation portion 62. The irradiation shaft 22 including a distal shaft 24 is fixed to the casing 61. When the movement operation portion 62 moves with respect to the casing 61, the irradiation shaft 22 does not move, and the main shaft 21 and the distal structure portion 30 move with respect to the casing 61. The casing 61 and the movement operation portion 62 includes a fixing element, and by switching a state of the fixing element, whether the movement operation portion 62 is slidable with respect to the casing 61 can be adjusted. A distal portion of the main shaft 21 is fixed to a proximal portion of the distal structure portion 30.

The main shaft 21 preferably has a certain degree of rigidity such that an operator can hold the movement operation portion 62 or the operation portion 60 to push the main shaft 21 to a desired position. A constituent material from which the main shaft 21 may be fabricated is not particularly limited, and includes: a metal represented by stainless steel, aluminum, titanium alloys, tin, magnesium alloys, or the like; a resin represented by polyetheretherketone (PEEK), polyamide, acrylonitrile butadiene styrene (ABS), polycarbonate, polyacetal, polyimide; or the like. A length of the main shaft 21 in an axial direction is not particularly limited, and is, for example, 100 mm to 400 mm.

The irradiation shaft 22 is a tubular member capable of accommodating the irradiation unit 50 therein, and is capable of transmitting light outward from the irradiation unit 50. A part of the irradiation shaft 22 is disposed inside the main shaft 21 and the distal structure portion 30. A distal portion of the irradiation shaft 22 extends toward the distal side relative to the main shaft 21 and the distal structure portion 30 (i.e., a distal portion of the irradiation shaft 22 extends distally beyond the main shaft 21 and the distal structure portion 30). The portion of the irradiation shaft 22 that protrudes from or distally beyond the distal structure portion 30 toward the distal side is a distal shaft 24. The distal shaft 24 is a portion to be inserted from the external uterine ostium O into the cervical canal CC in order to emit light from an inside of the cervical canal CC to the cervix U (see FIG. 12). A proximal portion of the irradiation shaft 22 extends toward the proximal side relative to the main shaft 21 and the operation portion 60 (i.e., the proximal portion of the irradiation shaft 22 extends proximally beyond the proximal end of the main shaft 21 and the proximal end of the operation portion 60). An irradiation lumen 25 in which the irradiation unit 50 is movable is formed inside the irradiation shaft 22. The irradiation lumen 25 is closed at a most distal end of the irradiation shaft 22, and is opened at a most proximal end of the irradiation shaft 22. An insertion port 28 for receiving the irradiation unit 50 into the irradiation lumen 25 is disposed on a proximal side of the irradiation shaft 22.

The irradiation shaft 22 is formed of a transparent or translucent material capable of transmitting light having a wavelength emitted by the irradiation unit 50 accommodated therein. A constituent material from which the irradiation shaft 22 may be fabricated is not particularly limited, and includes: a resin represented by polymethyl methacrylate, polyethylene terephthalate, polycarbonate, polytetrafluoroethylene, or the like; glass; or the like. It is more preferable that a material for the distal shaft 24 has elasticity and has a physical property allowing the distal shaft 24 to be deformed while being bent along a cervical canal after being inserted into the cervical canal. Accordingly, it is possible to cope with individual differences in a shape of the cervical canal, and it is possible to reduce a burden on an inner surface of the cervical canal and to further improve adhesion to the inner surface of the cervical canal. An outer diameter of the irradiation shaft 22 (the distal shaft 24) is not particularly limited, and is, for example, 0.5 mm to 6 mm. A length of the distal shaft 24 in the axial direction is not particularly limited, and is, for example, 10 mm to 50 mm. At least the distal shaft 24 of the irradiation shaft 22 may have a function of diffusing light. Therefore, similar to the distal structure portion 30 which will be described in detail later, the distal shaft 24 may contain scatterers in at least a part of the constituent material, may have multiple irregularities formed on an inner surface or an outer surface thereof, or may have a multi-layer structure in which materials having different refractive indexes are joined by a surface on which multiple irregularities are formed. The distal shaft 24 may be formed linearly, or may be curved to facilitate passing the distal shaft 24 through the cervical canal CC which is inclined with respect to the vagina V. The irradiation shaft 22 is formed rigidly, substantially rigidly, or flexibly.

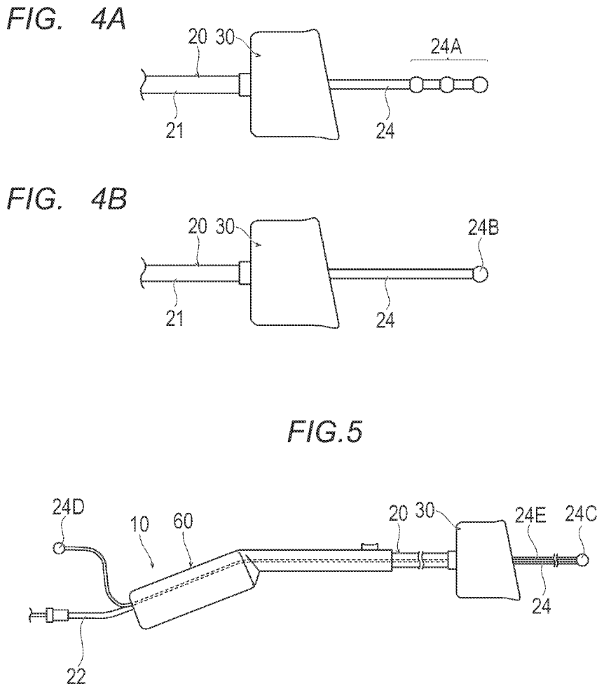

A shape of the distal shaft 24 is not particularly limited. For example, as in a first modification illustrated in FIG. 4A, the distal shaft 24 may include irregular structures 24A arranged in the axial direction (axially spaced-apart from one another). Accordingly, when inserting the distal shaft 24 from the external uterine ostium O into the cervical canal CC, the operator can easily grasp or understand a length of insertion of the distal shaft 24 into the cervical canal CC by visually checking the irregular structure 24A. When inserting the irregular structure 24A from the external uterine ostium O into the cervical canal CC, the operator can easily grasp or understand the length of insertion of the distal shaft 24 into the cervical canal CC based on a change in sensation received by a hand holding the operation portion 60. As a structure that facilitates visual checking, the distal shaft 24 may have a line, a notch, or the like serving as a scale. The distal shaft 24 may have physical properties that change along the axial direction such that the sensation received by the hand of the operator changes when the operator inserts the distal shaft 24 from the external uterine ostium O into the cervical canal CC. For example, the distal shaft 24 may be configured to decrease in rigidity toward the distal direction, or may have relatively high-rigidity portions and relatively low-rigidity portions arranged alternately.

As in a second modification illustrated in FIG. 4B, the distal shaft 24 may include a distal portion provided with one large-diameter portion 24B having a larger outer diameter than the adjoining portion of the distal shaft 24. Accordingly, after inserting the distal shaft 24 from the external uterine ostium O into the cervical canal CC, the operator can easily grasp or understand, based on the change in the sensation received by the hand holding the operation portion 60, that the large-diameter portion 24B crosses an internal cervical ostium I and reaches a uterine cavity UC. For example, the operator can retract the operation portion 60 and bring the large-diameter portion 24B into contact with the internal cervical ostium I after the large-diameter portion 24B crossed the internal cervical ostium I. Therefore, the distal shaft 24 including the large-diameter portion 24B is effective when it is desired to accurately position the distal portion of the distal shaft 24 with respect to the internal cervical ostium I, or when it is desired to reliably pass the distal portion of the distal shaft 24 through the internal cervical ostium I. A position of the large-diameter portion 24B is not limited to a most distal end of the distal shaft 24.

As in a third modification illustrated in FIG. 5, the distal shaft 24 may include a distal portion provided with a bag-shaped first balloon 24C that is flexibly deformable. The first balloon 24C communicates with a bag-shaped second balloon 24D disposed on the operation portion 60 by a tube 24E. A fluid is sealed in the first balloon 24C, the second balloon 24D, and the tube 24E. Accordingly, when the distal shaft 24 enters the cervical canal CC from the external uterine ostium O, the first balloon 24C is compressed, the fluid inside the first balloon 24C moves toward the second balloon 24D, and the second balloon 24D is inflated greatly. Accordingly, the operator can easily grasp or understand, by viewing the second balloon 24D, that the distal shaft 24 including the first balloon 24C enters the cervical canal CC. When the first balloon 24C crosses the internal cervical ostium I, the first balloon 24C is inflated due to restoring force per se, the fluid inside the second balloon 24D moves toward the first balloon 24C, and the second balloon 24D becomes small. Accordingly, the operator can easily understand or grasp, by viewing the second balloon 24D, that the distal shaft 24 including the first balloon 24C crosses the internal cervical ostium I.

The operator may insert the distal shaft 24 from the external uterine ostium O into the cervical canal CC in a state in which the irradiation unit 50 disposed inside the distal shaft 24 is caused to emit light. Light emitted from a portion of the distal shaft 24 inserted into the cervical canal CC is invisible (not visible) to the operator. Therefore, the operator can easily visually grasp the length of insertion of the distal shaft 24 into the cervical canal CC. In this case, even if the distal shaft 24 is not provided with the irregular structure 24A or the large-diameter portion 24B, the operator can visually grasp the length of insertion of the distal shaft 24 into the cervical canal CC.

As illustrated in FIGS. 3 and 11 to 13, the distal structure portion 30 is a member that is disposed on a proximal side of the distal shaft 24 to be inserted into the cervical canal CC, and is capable of being inserted into the vagina V and emitting light in a wide range of the vagina V. The distal structure portion 30 is larger the main shaft 21 in a radial direction of the main shaft 21. That is, as illustrated in, for example, FIG. 3, the distal structure portion 30 is configured so that the outermost surface of the distal structure portion 30 is radially outwardly of the outer surface of the main shaft 21 (and radially outwardly of the outer surface of the distal shaft 24). The distal structure portion 30 can transmit outward light emitted from the irradiation unit 50 disposed in the irradiation lumen 25 that passes through an inside of the distal structure portion 30. Therefore, the distal structure portion 30 is formed of a transparent or translucent material capable of transmitting light having a wavelength emitted by the irradiation unit 50.

As illustrated in FIG. 3, the distal structure portion 30 is cup-shaped with a recessed portion 31 formed on a distal side thereof. The distal structure portion 30 includes a connection portion 32 connected to the main shaft 21, a diameter expanded portion 33 extending radially outward from the connection portion 32, and a tubular wall portion 34 surrounding the recessed portion 31. As shown in FIG. 3, this embodiment of the distal structure portion 30 is configured so that the tubular wall portion 34 axially overlaps a portion of the distal shaft 24. The connection portion 32 is formed with a through hole 35 that penetrates in a manner of allowing the distal shaft 24 to move in the axial direction. The diameter expanded portion 33 is formed in a substantially disk shape, but the shape of the diameter expanded portion 33 is not particularly limited. The diameter expanded portion 33 is formed to be substantially perpendicular to central axis of the main shaft 21, but may be formed to be inclined. It is preferable that a thickness of the diameter expanded portion 33 decreases radially outward. Accordingly, light incident from an inner wall surface of the through hole 35 into the material for the distal structure portion 30 can be guided radially outward through the material while being reflected by a surface of the material. The through hole 35 may extend from the distal structure portion 30 in a proximal direction of the main shaft 21, and a length of the through hole 35 is more preferably equal to or longer than that of a light-emitting unit 52. The diameter expanded portion 33 may be formed with a constant thickness.

The wall portion 34 has a substantially tubular shape and surrounds the recessed portion 31. A proximal portion of the wall portion 34 is connected to a radially outer site or portion of the diameter expanded portion 33. The wall portion 34 extends in a cylindrical shape from a connection site with the diameter expanded portion 33 in the distal direction. It is preferable that a thickness of the wall portion 34 decreases toward the distal side. Accordingly, the wall portion 34 can propagate light propagated from the diameter expanded portion 33 to the proximal portion of the wall portion 34 through a material for the diameter expanded portion 33, to the distal side through the material while reflecting the light on a surface of the material. The wall portion 34 may be formed with a constant thickness.

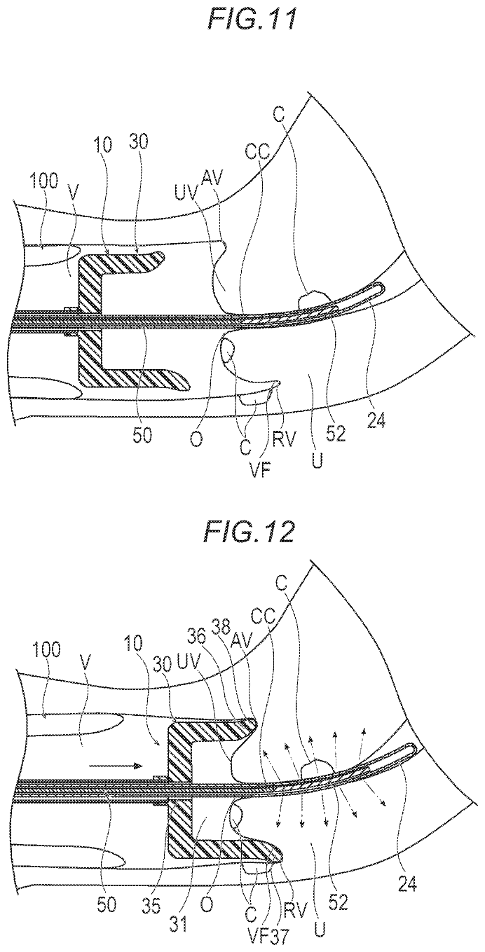

The distal portion of the wall portion 34 is formed with a cup distal portion 36. The cup distal portion 36 expands in the distal direction. That is, an inner diameter and an outer diameter of the cup distal portion 36 increase in the distal direction. Since the wall portion 34 includes the cup distal portion 36 that expands toward the distal side, the uterine vagina UV can be easily received in the recessed portion 31 (see FIG. 11). This enables the cup distal portion 36 to reach the vaginal vault VF, which is difficult to access, or to the vicinity of the vaginal vault VF. A surface where a most distal end of the cup distal portion 36 is positioned is inclined at an angle θ of less than 90° with respect to a plane perpendicular to the central axis of the through hole 35. Therefore, the cup distal portion 36 is formed with a protruding portion 37 that protrudes most in the distal direction at a part of the cup distal portion 36 in a peripheral direction. The cup distal portion 36 is formed with a depression portion 38 having a smallest protruding amount in the distal direction on an opposite side of the protruding portion 37 in the peripheral direction.

A length L1 from a proximal surface of the distal structure portion 30 to the depression portion 38 is, for example, 5 mm to 20 mm. A length L2 from the proximal surface of the distal structure portion 30 to the protruding portion 37 is, for example, 10 mm to 30 mm.

By disposing the depression portion 38 on an anterior vaginal vault AV side near a vaginal introitus and disposing the protruding portion 37, which is on an opposite side of the depression portion 38, on a posterior vaginal vault RV side far from the vaginal introitus, the cup distal portion 36 can be brought close to the entire vaginal vault VF including the anterior vaginal vault AV and the posterior vaginal vault RV. Therefore, light can be effectively emitted to a range where light is difficult to reach, including the posterior vaginal vault RV and the anterior vaginal vault AV.

A constituent material from which the distal structure portion 30 may be fabricated is not particularly limited as long as the constituent material has a certain degree of rigidity and can transmit light having a wavelength emitted from the irradiation unit 50, and is, for example, silicone, polyamide, polymethyl methacrylate, polyethylene terephthalate, polycarbonate, polytetrafluoroethylene, urethane, and the like, and combinations thereof. A maximum outer diameter of the distal structure portion 30 is not particularly limited, and is, for example, 20 mm to 50 mm. A length of the distal structure portion 30 in the axial direction is not particularly limited, and is, for example, 5 mm to 30 mm.

The distal structure portion 30 may have a structure that scatters the light received from the irradiation unit 50 inside the distal structure portion 30. The inside of the distal structure portion 30 means an inside of the through hole 35 or an inside of the recessed portion 31. The inside of the recessed portion 31 is on the distal side relative to the through hole 35, on the proximal side relative to the most distal end of the distal structure portion 30, and on an inner side in the radial direction relative to an inner peripheral surface of the wall portion 34. Light emitted from the inside of the through hole 35 enters the material for the distal structure portion 30 from the through hole 35, and is propagated radially outward through the material of the diameter expanded portion 33. Light emitted inside the recessed portion 31 of the distal structure portion 30 can enter the inside of the material for the distal structure portion 30 from an inner surface of the recessed portion 31 (for example, a surface of the diameter expanded portion 33 on the distal side, or the inner peripheral surface of the wall portion 34). Accordingly, the cup per se emits light by the light received from the irradiation unit 50. Therefore, the treatment apparatus 10 can emit light to a wide range through the distal structure portion 30 even in a range that cannot be directly irradiated with the light from the irradiation unit 50.

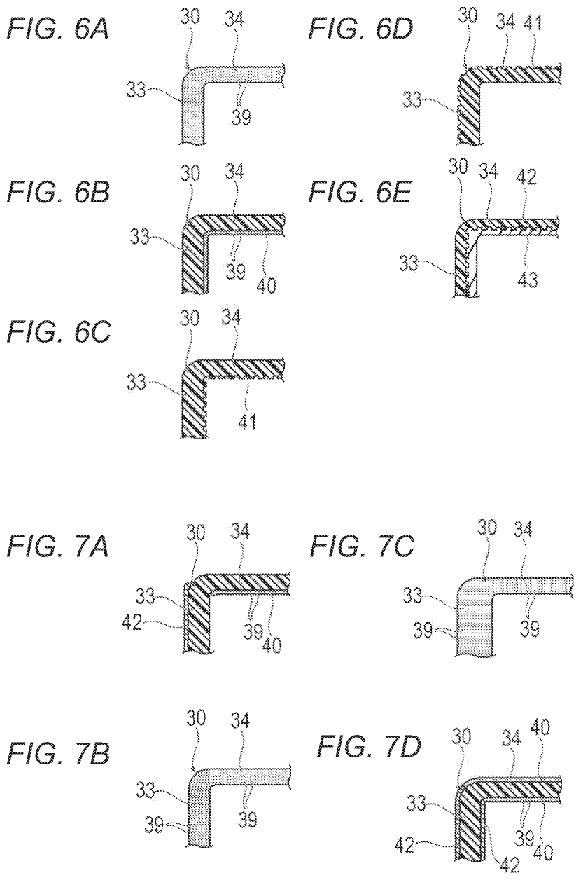

The distal structure portion 30 may have a structure that scatters light. Accordingly, the cup per se emits light by the light received from the irradiation unit 50. For example, as in a fourth modification illustrated in FIG. 6A, the distal structure portion 30 may contain scatterers 39 inside the material. The scatterer 39 may be implemented by known materials, and may be fine particles of titanium oxide, styrene, silicone, or the like. As in a fifth modification illustrated in FIG. 6B, the distal structure portion 30 may include, on an inner surface thereof (a surface on a recessed portion 31 side), a scatterer coat 40 including the scatterer 39. The scatterer coat 40 is coated by mixing the scatterer 39 with a coat substrate having a refractive index different from that of the scatterer 39. As a structure for scattering light, the distal structure portion 30 may include, on the inner surface thereof, multiple minute irregular portions 41, as in a sixth modification illustrated in FIG. 6C. As a structure for scattering light, the distal structure portion 30 may include, on an outer surface thereof, multiple minute irregular portions 41, as in a seventh modification illustrated in FIG. 6D. When the irregular portions 41 on the outer surface of the distal structure portion 30 (a surface on a side opposite to the recessed portion 31 side) come into contact with a living body (an organ) such as the uterine vagina UV or the vagina V, light emitted from the material for the distal structure portion 30 is easily transmitted into the living body without being reflected by the irregular portions 41, and an amount of light in the material for the distal structure portion 30 is decreased. Therefore, by providing a detection unit 90 (see FIG. 14) capable of detecting the amount of light in the material for the distal structure portion 30, it is possible to determine that the distal structure portion 30 is in close contact with the living body. In order to facilitate the transmission of the light to the living body when the irregular portions 41 of the distal structure portion 30 come into contact with the living body, a refractive index of the distal structure portion 30 is preferably higher than a refractive index of air, and equal to or lower than a refractive index of the living body, and is, for example, about higher than 1.0 to 1.5. As in an eighth modification illustrated in FIG. 6E, the distal structure portion 30 may have a structure in which a first layer 42 and a second layer 43 having different refractive indexes are joined by a surface having irregularities.

The distal structure portion 30 may have a structure that increases an irradiation intensity in a specific direction. For example, it is preferable that the distal structure portion 30 does not emit light in the proximal direction and emits light in the radial direction and the distal direction. Accordingly, it is possible to increase an intensity of light that can be emitted from the distal structure portion 30 to the tumor cells C of the cervix U or the vagina V close to the cervix U. The structure for increasing the irradiation intensity in the specific direction is, for example, a structure in which light is less likely to leak outward from a proximal side of the distal structure portion 30. For example, the distal structure portion 30 may include, on an outer surface of the diameter expanded portion 33, a reflector coat 42 formed of a reflector that reflects light, as in a ninth modification illustrated in FIG. 7A. The reflector may be disposed inside the material for the distal structure portion 30 or on the inner surface of the distal structure portion 30. As in a 10th modification illustrated in FIG. 7B, the scatterer 39 may be contained inside the material for the distal structure portion 30, and a concentration of the scatterer 39 in the diameter expanded portion 33 may be set to be higher than a concentration of the scatterer 39 in the wall portion 34. As in an 11th modification illustrated in FIG. 7C, the scatterer 39 may be contained in the material for the distal structure portion 30, and a thickness of the diameter expanded portion 33 may be thicker than a thickness of the wall portion 34. As in a 12th modification illustrated in FIG. 7D, the distal structure portion 30 may include the reflector coat 42 on both surfaces of the diameter expanded portion 33, and may include the scatterer coat 40 on both surfaces of the wall portion 34. Accordingly, light that enters the material for the distal structure portion 30 from the through hole 35 can be propagated to the wall portion 34 while being reflected by the reflector coat 42 on both surfaces of the diameter expanded portion 33. Then, the light in the material for the wall portion 34 can be scattered by the scatterer coat 40 on both surfaces of the wall portion 34, and can be uniformly emitted outward.

The distal structure portion 30 may be formed in various shapes. It is preferable that the distal structure portion 30 is appropriately selectable according to a shape of the uterine vagina UV, the vaginal vault VF, or the vagina V of the patient.

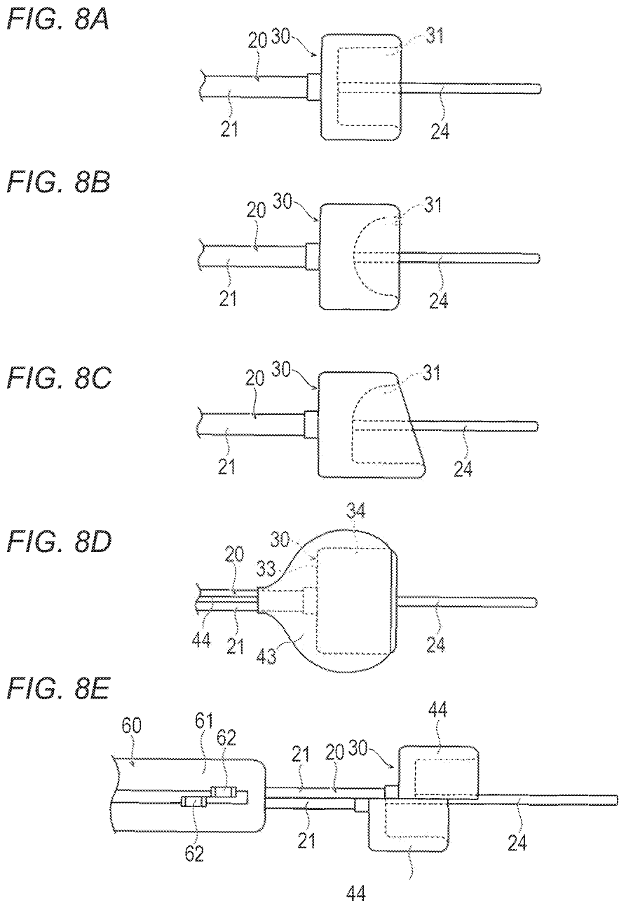

As in a 13th modification illustrated in FIG. 8A, a surface of the distal structure portion 30 on the distal side may be substantially perpendicular to the center axis of the main shaft 21 (the central axis of the through hole 35). As in a 14th modification illustrated in FIG. 8B, the recessed portion 31 may be formed in a smooth arc shape in a cross section passing through the center axis of the main shaft 21. As in a 15th modification illustrated in FIG. 8C, the recessed portion 31 may be formed in a partially smooth arc shape in the cross section passing through the center axis of the main shaft 21, with the other part the recessed portion 31 being substantially perpendicular to the center axis of the main shaft 21.

As in a 16th modification illustrated in FIG. 8D, the distal structure portion 30 may include a balloon 43 that covers an outer peripheral surface of the wall portion 34, an outer peripheral surface of the main shaft 21, and a proximal surface of the diameter expanded portion 33. The balloon 43 can be inflated by being supplied with a fluid via a supply tube 44 extending from the operation portion 60. By inflating the balloon 43, the distal structure portion 30 can be brought into close contact with the uterine vagina UV, the vaginal vault VF, and the vagina V. The balloon 43 may cover only the outer peripheral surface of the wall portion 34, may cover only the outer peripheral surface of the main shaft 21, or may cover the proximal surface of the diameter expanded portion 33.

As in a 17th modification illustrated in FIG. 8E, the distal structure portion 30 may be divided into two or more (two in the 17th modification) sub-distal structure portions 44. Each sub-distal structure portion 44 is connected to a movement operation portion 62 that is independently movable, and is movable independently along the center axis. Therefore, for example, in order to secure a visual field, the operator can position one sub-distal structure portion 44 at the uterine vagina UV or the vaginal vault VF, and then position the other sub-distal structure portion 44 at the uterine vagina UV or the vaginal vault VF.

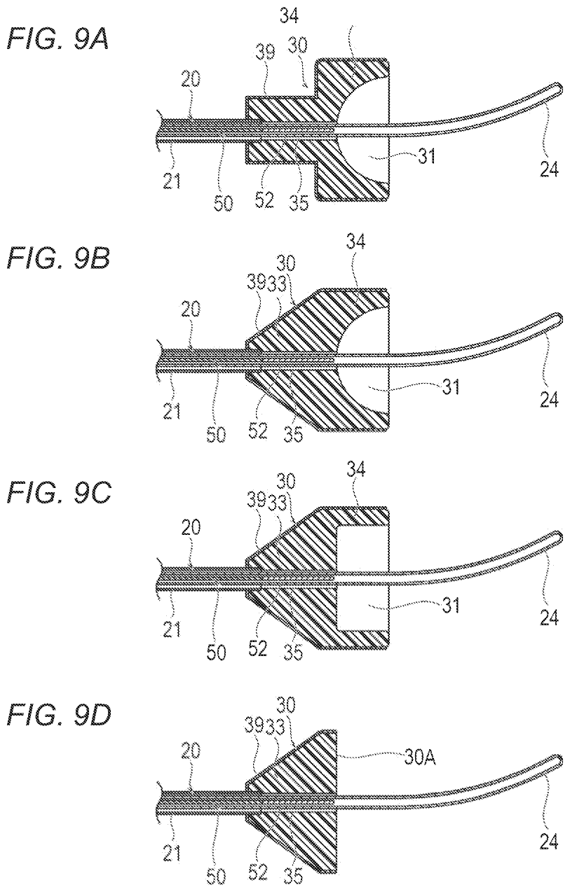

As in an 18th modification illustrated in FIG. 9A, the through hole 35 of the distal structure portion 30 may be elongated in the axial direction of the distal structure portion 30. The length of the through hole 35 of the distal structure portion 30 in the axial direction is not limited, and is preferably equal to or longer than the length of the light-emitting unit 52, which is a light emitting portion of the light output device 80 that will be described later, in the axial direction. Accordingly, the light emitted from the light-emitting unit 52 can be input to the distal structure portion 30 without waste. A surface of the distal structure portion 30 that faces the proximal side and a surface of the distal structure portion 30 that faces outward in the radial direction are preferably coated with a reflector coat 39. A surface of the distal structure portion 30 that faces the distal side and the inner surface of the recessed portion 31 of the distal structure portion 30 are not coated with the reflector coat 39. The surface of the distal structure portion 30 that faces the distal side and the inner surface of the recessed portion 31 of the distal structure portion 30 may be coated with a scatterer coat 36. Accordingly, the light emitted from the light-emitting unit 52 can be input to the distal structure portion 30 with a small loss and output in a desired direction. The light-emitting unit 52 emits light inside the distal structure portion 30, and the excitation light emitted from the distal structure portion 30 is emitted only in the distal direction (a direction in which the external uterine ostium O and the uterine vagina UV are present with respect to the distal structure portion 30). Therefore, a treatment effect on the external uterine ostium O and the uterine vagina UV can be improved.

As in a 19th modification illustrated in FIG. 9B, the diameter expanded portion 33 of the distal structure portion 30 may be formed to have an outer diameter increasing in the distal direction. Other configurations are the same as those of the 18th modification. That is, the length of the through hole 35 of the distal structure portion 30 in the axial direction is preferably equal to or longer than the length of the light-emitting unit 52 in the axial direction. Accordingly, the light emitted from the light-emitting unit 52 can be input to the distal structure portion 30 without waste. The light input to the distal structure portion 30 is effectively reflected in the distal direction by the reflector coat 39 coated on an inclined outer surface of the diameter expanded portion 33. Therefore, the treatment effect on the external uterine ostium O and the uterine vagina UV can be further improved.

A 20th modification illustrated in FIG. 9C is different from the 19th modification only in a shape of the recessed portion 31 of the distal structure portion 30. The shape of the recessed portion 31 is not particularly limited. Therefore, the recessed portion 31 of the 19th modification illustrated in FIG. 9B is formed in a smooth arc shape in the cross section passing through the center axis of the main shaft 21, but the recessed portion 31 of the 20th modification illustrated in FIG. 9C is formed such that an inner diameter thereof is substantially constant in the axial direction.

A 21st modification illustrated in FIG. 9D is different from the 20th modification only in that the distal structure portion 30 is not formed with the recessed portion 31 and the wall portion 34. A distal surface 30A of the distal structure portion 30 that faces the distal side is formed by, for example, a flat surface, but the distal surface 30A may not be a flat surface, and may protrude toward the distal side, for example. The distal surface 30A may or may not be coated with the scatterer coat 36. The light-emitting unit 52 emits light inside the distal structure portion 30, and the excitation light emitted from the distal structure portion 30 is emitted only in the distal direction (the direction in which the external uterine ostium O and the uterine vagina UV are present with respect to the distal structure portion 30). Therefore, the treatment effect on the external uterine ostium O and the uterine vagina UV can be improved.

As illustrated in FIGS. 1 and 3, the irradiation unit 50 is elongated, and includes at least one optical fiber 51 that propagates light. The irradiation unit 50 includes, at a distal portion thereof, the light-emitting unit 52 that emits light outward. A proximal portion of the irradiation unit 50 is connectable to the light output device 80 which outputs light. The irradiation unit 50 can receive near-infrared rays from the light output device 80, propagate the near-infrared rays to the light-emitting unit 52, and emit the near-infrared rays from the light-emitting unit 52. The irradiation unit 50 may be formed by an optical waveguide other than the optical fiber.

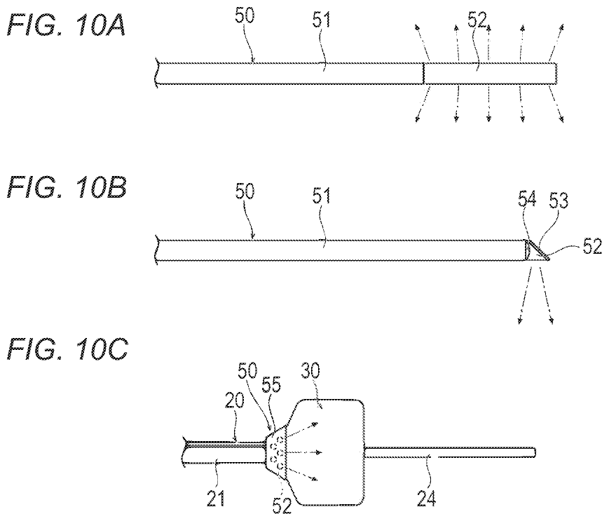

As illustrated in FIG. 10A, the light-emitting unit 52 is a cylindrical diffuser that is connected to a cut stump (cut end) of the optical fiber 51 and diffuses or scatters light received from the optical fiber 51. The diffuser may be formed integrally with the optical fiber 51 by processing a surface or an inside of the optical fiber 51. The light-emitting unit 52 may be the cut stump (cut end) of the optical fiber 51. In this case, it is preferable to provide a plurality of optical fibers 51 to emit light with a wide irradiation angle. The light-emitting unit 52 may be formed by a mirror 53 and/or a lens 54 disposed at the cut stump of the optical fiber 51, as in a 22nd modification illustrated in FIG. 10B. By forming the light-emitting unit 52 by the mirror 53 and/or the lens 54, the irradiation angle of light can be widened. By rotating the optical fiber 51, the light-emitting unit 52 can emit light in a wider range.

The light-emitting unit 52 may not be disposed inside the shaft portion 20 or may not be disposed inside the distal structure portion 30. For example, as in a 23rd modification illustrated in FIG. 10C, the irradiation unit 50 may include an irradiation auxiliary unit 55 that surrounds the shaft portion 20 on the proximal side of the distal structure portion 30, and the light-emitting unit 52 may be disposed in the irradiation auxiliary unit 55. The irradiation auxiliary unit 55 has an inner peripheral surface that expands toward the distal direction in a manner of covering a part of the surface of the diameter expanded portion 33 on the proximal side. The light-emitting unit 52 is disposed on the inner peripheral surface. The light-emitting unit 52 is, for example, the stump of the optical fiber, the diffuser, the mirror, the lens, and an LED that emits light by electric power. When the light-emitting unit 52 of the irradiation auxiliary unit 55 emits light, light is emitted from the proximal side of the distal structure portion 30 to the inside of the distal structure portion 30. Accordingly, the distal structure portion 30 can emit light substantially as a whole by receiving light from the light-emitting unit 52 of the irradiation auxiliary unit 55. The light-emitting unit 52 provided in the irradiation auxiliary unit 55 may be used together with the irradiation unit 50 provided in the irradiation lumen 25.

The operation portion 60 is a part to be held and operated by the operator, as illustrated in FIG. 1. The proximal portion of the main shaft 21 is fixed to the operation portion 60. The irradiation shaft 22 is led out from a proximal portion of the operation portion 60. The irradiation shaft 22 may be fixed at the proximal portion of the operation portion 60. The operation portion 60 is formed to be bent from a distal portion toward the proximal portion to easily secure the visual field of the operator in the vagina V when inserting the distal structure portion 30 and the distal shaft 24 from the vaginal introitus. A configuration of the operation portion 60 is not particularly limited.

The light output device 80 can output light having any wavelength to the optical fiber 51 of the irradiation unit 50 with any intensity (power) or energy. The light output device 80 outputs near-infrared rays having a wavelength of, for example, 660 nm to 740 nm, to the optical fiber 51 such that light can be emitted at an intensity (power) of, for example, 1 mW to 5 W, and an energy of, for example, 1 Jcm-2 to 50 Jcm-2.

Next, the treatment method using the treatment apparatus 10 according to the embodiment will be described.

First, the antibody-photosensitive substance is administered intravenously. Approximately 12 to 36 hours after the intravenous administration, as illustrated in FIG. 11, the operator opens the vaginal introitus by using a vaginal speculum 100, and inserts the treatment apparatus 10 from the vaginal introitus with the distal structure portion 30 being retracted toward the proximal side with respect to the distal shaft 24 into the vagina V. At this time, the operator inserts the treatment apparatus 10 starting from the distal shaft 24. Next, the operator inserts the distal portion of the distal shaft 24 from the external uterine ostium O into the cervical canal CC while visually checking the distal portion of the distal shaft 24. At this time, since the distal structure portion 30 is retracted toward the proximal side with respect to the distal shaft 24, the operator can easily insert the distal shaft 24 into the cervical canal CC. Therefore, the operator can easily position the distal shaft 24 at a desired position with respect to the cervix U.

Next, the operator pushes the movement operation portion 62, and presses the distal structure portion 30 toward the uterine vagina UV, as illustrated in FIG. 12. Since the distal shaft 24 inserted from the external uterine ostium O into the cervical canal CC passes through the through hole 35 formed in a bottom surface of the recessed portion 31, the uterine vagina UV positioned around the external uterine ostium O enters the recessed portion 31. Therefore, the cup distal portion 36, which is positioned on a radially outer side of the distal structure portion 30 and protrudes in the distal direction, approaches the vaginal vault VF. At this time, the depression portion 38 of the cup distal portion 36 can come into contact with or approach the anterior vaginal vault AV near the vaginal introitus. The protruding portion 37 of the cup distal portion 36 can come into contact with or approach the posterior vaginal vault RV far from the vaginal introitus. At least a part of the ring-shaped cup distal portion 36 preferably abuts against the vaginal vault VF. Accordingly, the distal structure portion 30 can be positioned with respect to the cervix U and the vagina V. The operator may move the distal shaft 24 together with the distal structure portion 30 when positioning the distal structure portion 30. In this case, the distal structure portion 30 and the distal shaft 24 are simultaneously positioned with respect to the cervix U and the vagina V.

Next, the operator disposes the light-emitting unit 52 of the irradiation unit 50 inside the distal shaft 24. Thereafter, the operator operates the light output device 80 to supply near-infrared rays to the irradiation unit 50. Accordingly, the light-emitting unit 52 inside the distal shaft 24 can effectively emit the near-infrared rays to the tumor cells C positioned in the cervix U. An irradiation direction of the near-infrared rays from the light-emitting unit 52 includes a direction substantially perpendicular to an center axis of the distal shaft 24. Therefore, the light-emitting unit 52 can effectively emit the near-infrared rays from the cervical canal CC to the tumor cells C positioned in the cervix U. The operator may cause the near-infrared rays to be emitted while moving the light-emitting unit 52 inside the distal shaft 24.

When the near-infrared rays are emitted, the near-infrared rays reach the antibody-photosensitive substance bound to the tumor cells C in the cervix U. Accordingly, a chemical change occurs in the antibody-photosensitive substance that receives the near-infrared rays, which serve as the excitation light, and then a structural change occurs in the antibody-photosensitive substance, which generates holes in the cell membranes. Accordingly, the tumor cells C irradiated with the near-infrared rays are destroyed.

When the operator determines that the tumor cells C are sufficiently destroyed or a predetermined time passes, the operator stops emitting the near-infrared rays.

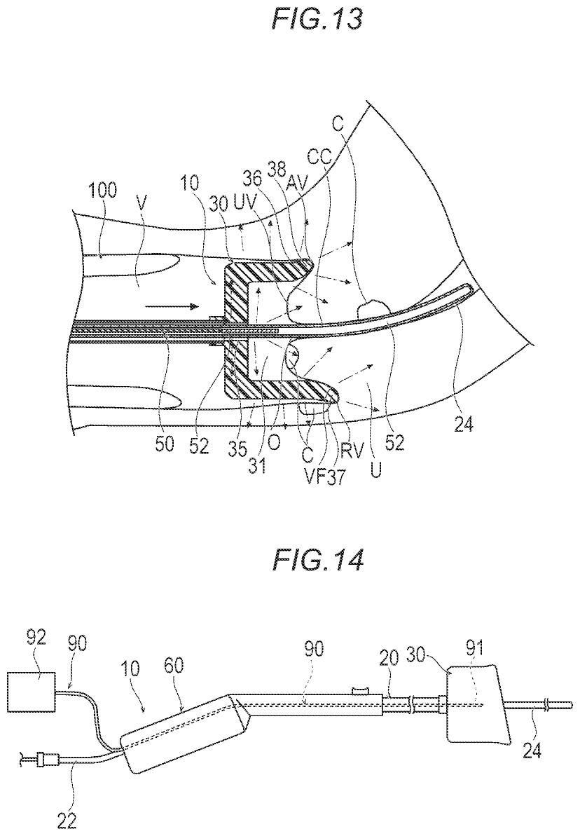

Next, as illustrated in FIG. 13, the operator pulls the irradiation unit 50 and moves the light-emitting unit 52 inside the distal structure portion 30 in a state in which the distal shaft 24 and the distal structure portion 30 are held. The light-emitting unit 52 is disposed, for example, inside the through hole 35 and the recessed portion 31. Thereafter, the operator operates the light output device 80 to supply the near-infrared rays to the irradiation unit 50. Accordingly, the entire distal structure portion 30 that receives the light from the light-emitting unit 52 emits light. That is, the light-emitting unit 52 disposed inside the through hole 35 reach the distal structure portion 30 from the through hole 35, and the light-emitting unit 52 disposed inside the recessed portion 31 reach the distal structure portion 30 from the recessed portion 31. A part of the near-infrared rays that reaches the distal structure portion 30 is transmitted through the distal structure portion 30, and a part of the near-infrared rays that reaches the distal structure portion 30 is scattered or reflected by the distal structure portion 30, and then emitted to a wide range. When the distal structure portion 30 includes a structure that increases an irradiation intensity in the distal direction (see FIGS. 7A to 7D), the near-infrared rays are emitted in a direction substantially perpendicular to a center axis of the irradiation shaft 22 and the distal direction. Therefore, the light-emitting unit 52 and the distal structure portion 30 can effectively emit the near-infrared rays to the tumor cells C positioned mainly at the external uterine ostium O, the uterine vagina UV, the vaginal vault VF, and the site that is near the vaginal vault VF and is on the vaginal introitus side relative to the vaginal vault VF of the vagina V. Multiple of folds are present in a vaginal wall on the vaginal introitus side relative to the vaginal vault VF of the vagina V, and by disposing the cup distal portion 36 near the vaginal vault VF, incident angles of the near-infrared rays to the vaginal wall become small. Therefore, reflection of light can be reduced as much as possible, and the near-infrared rays can be effectively emitted to the tumor cells C. The operator may cause the near-infrared rays to be emitted while moving the light-emitting unit 52 inside the distal structure portion 30. The operator may cause the near-infrared rays to be emitted while alternately moving the light-emitting unit 52 between the inside of the distal structure portion 30 and the inside of the distal shaft 24. When the light-emitting unit 52 is elongated in the axial direction and can emit light simultaneously from both the distal shaft 24 and the distal structure portion 30, the operator does not need to move the light-emitting unit 52 between the distal shaft 24 and the distal structure portion 30.

When the near-infrared rays are emitted, the near-infrared rays reach the antibody-photosensitive substance bound to the tumor cells C mainly in the external uterine ostium O, the uterine vagina UV, the vaginal vault VF, and the site that is near the vaginal vault VF and is on the vaginal introitus side relative to the vaginal vault VF of the vagina V. Accordingly, the chemical change occurs in the antibody-photosensitive substance that receives the near-infrared rays, which serve as the excitation light, and then the structural change occurs in the antibody-photosensitive substance, which generates holes in the cell membranes. Accordingly, the tumor cells C irradiated with the near-infrared rays are destroyed.

As necessary, the operator can repeatedly perform treatment of emitting the near-infrared rays by appropriately moving the light-emitting unit 52 to an appropriate position (inside the through hole 35 and/or the recessed portion 31) while moving the distal structure portion 30 in the vagina V by operating the entire movement operation portion 62 or the operation portion 60.

When the operator determines that the tumor cells C are sufficiently destroyed or a predetermined time passes, the operator stops emitting the near-infrared rays. Thereafter, the operator retracts the distal structure portion 30, and draws the treatment apparatus 10 out of the cervical canal CC and the vagina V. Accordingly, this treatment method ends.

As described above, the treatment apparatus 10 according to the present embodiment is the treatment apparatus 10 configured to irradiate the antibody-photosensitive substance bound to the tumor cell C of cervical cancer with excitation light. The treatment apparatus 10 including: the main shaft 21 including the distal portion and the proximal portion; the distal structure portion 30 disposed on the distal side of the main shaft 21 and formed to be larger than the main shaft 21 in the radial direction of the main shaft 21; the distal shaft 24 protruding from the distal structure portion 30 toward the distal side; and at least one irradiation unit 50 configured to emit the excitation light of the antibody-photosensitive substance from the distal shaft 24 and the distal structure portion 30.

According to the treatment apparatus 10 described above, the excitation light can be effectively emitted to the antibody-photosensitive substance bound to the tumor cells C in a wide range including the cervix U in a state in which the distal shaft 24 is inserted into the cervical canal CC and the distal structure portion 30 is inserted into the vicinity of the external uterine ostium O of the vagina V. Therefore, this treatment apparatus 10 can improve the treatment effect of cancer in a wide range including at least a part of the cervix U.

The treatment apparatus 10 is formed with the irradiation lumen 25 communicating with the inside of the through hole 35 and the inside of the distal shaft 24, and configured to movably accommodate the irradiation unit 50, the through hole 35 penetrating from the distal side to the proximal side of the distal structure portion 30. Accordingly, even if only one irradiation unit 50 is provided, the excitation light can be emitted from the distal shaft 24 and the distal structure portion 30, and thus a configuration of the treatment apparatus 10 can be simplified and operability can be improved. By moving the irradiation unit 50, a position where the excitation light is emitted can be appropriately adjusted, and thus the treatment effect can be improved.

The distal structure portion 30 has a cup shape with the recessed portion 31 formed on the distal side thereof. Accordingly, the excitation light can be effectively emitted to the antibody-photosensitive substance bound to the tumor cells CC in a wide range including the cervix U in a state in which the distal shaft 24 is inserted into the cervical canal CC and a site or portion surrounding the recessed portion 31 of the distal structure portion 30 is inserted into the vicinity of the vaginal vault VF. Therefore, this treatment apparatus 10 can improve the treatment effect of cancer in a wide range including at least a part of the cervix U.

The distal structure portion 30 includes the wall portion 34 surrounding the recessed portion 31 and protruding toward the distal side, and the wall portion 34 includes, at a part in the peripheral direction surrounding the recessed portion 31, the protruding portion 37 having a protruding amount in the distal direction larger than those of other portions. Accordingly, the wall portion 34 can be brought close to the vaginal vault VF. Therefore, the excitation light can be effectively emitted to the vicinity of the vaginal vault VF, which is difficult for light to reach, and the treatment effect can be improved.

The distal shaft 24 may be configured to emit the excitation light in the direction substantially perpendicular to the center axis of the distal shaft 24, and the distal structure portion 30 may be configured to emit the excitation light in a substantially distal direction. Accordingly, the excitation light can be emitted to the tumor cells CC of the cervix U from both the distal shaft 24 and the distal structure portion 30, and thus the treatment effect can be improved.

The distal structure portion 30 is configured to move relative to the main shaft 21 in the axial direction of the main shaft 21. Accordingly, the distal shaft 24 can be inserted into the cervical canal CC in a state in which the distal structure portion 30 is retracted toward the proximal side with respect to the main shaft 21 to secure the visual field. In a state in which the distal shaft 24 is maintained at an appropriate position of the cervical canal CC, the distal structure portion 30 can be moved and disposed at an appropriate position. Therefore, both the distal shaft 24 and the distal structure portion 30 can be accurately and easily disposed at appropriate positions of the cervical canal CC and the vagina V. Therefore, the excitation light can be emitted from the distal shaft 24 and the distal structure portion 30 to desired positions, and thus the treatment effect can be improved.

The treatment method according to the present embodiment is a treatment method for cervical cancer. The treatment method includes: a step of intravenously administering the antibody-photosensitive substance; a step of inserting the treatment apparatus 10 into the vagina V 12 to 36 hours after the intravenous administration, the treatment apparatus 10 including the distal structure portion 30 disposed on the distal side of the elongated main shaft 21 and the distal shaft 24 protruding from the distal structure portion 30 toward the distal side, and being configured to emit the excitation light of the antibody-photosensitive substance; a step of inserting the distal shaft 24 into the cervical canal CC; a step of inserting the distal structure portion 30 into the vagina V; a step of emitting the excitation light from the distal shaft 24 to a surrounding tissue; and a step of emitting the excitation light from the distal structure portion 30 to a surrounding tissue.

According to the treatment method described above, the distal shaft 24 can be inserted from an external uterine ostium into the cervical canal CC, and the distal structure portion 30 can be inserted into the vagina V (for example, the vicinity of the external uterine ostium O or the uterine vagina UV, or a position in contact with the external uterine ostium O or the uterine vagina UV), and thus by emitting the excitation light of the antibody-photosensitive substance from the distal shaft 24 and the distal structure portion 30, the excitation light can be effectively emitted to the antibody-photosensitive substance bound to the tumor cells CC in a wide range including the cervix U. Therefore, this treatment method can improve the treatment effect of cancer in a wide range including at least a part of the cervix U.

The distal structure portion 30 may have a cup shape with the recessed portion 31 formed on a distal side thereof, and in the step of inserting the distal structure portion 30 into the vagina V, at least a part of the distal structure portion 30 may be inserted into the vaginal vault VF. Accordingly, the excitation light can be effectively emitted to the antibody-photosensitive substance bound to the tumor cells CC in a wide range including the cervix U. Therefore, this treatment method can improve the treatment effect of cancer in a wide range including the cervix U.

In the step of emitting the excitation light from the distal shaft 24, the irradiation unit 50 configured to emit the excitation light may be disposed inside the distal shaft 24 to emit the excitation light from the irradiation unit 50, in the step of emitting the excitation light from the distal structure portion 30, the irradiation unit 50 may be disposed inside the distal structure portion to emit the excitation light from the irradiation unit 50, and the irradiation unit 50 may be moved between the distal shaft 24 and the distal structure portion 30 between the step of emitting the excitation light from the distal shaft 24 and the step of emitting the excitation light from the distal structure portion 30. Accordingly, with one irradiation unit 50, the excitation light can be emitted from the distal shaft 24 and the distal structure portion 30, and thus the configuration of the treatment apparatus 10 can be simplified and the operability can be improved. By moving the irradiation unit 50, a position where the excitation light is emitted can be appropriately adjusted, and thus the treatment effect can be improved. An order of emitting the excitation light is not limited. Therefore, the excitation light may be emitted from the distal shaft 24 first, or the excitation light may be emitted from the distal structure portion 30 first.

In this treatment method, the step of emitting the excitation light from the distal shaft 24 and the step of emitting the excitation light from the distal structure portion 30 may be performed simultaneously. Accordingly, this treatment method can simultaneously emit the excitation light from various positions and directions, and thus the treatment effect can be improved, and treatment can be efficiently performed in a short time.

The invention is not limited to the embodiments described above, and various modifications can be made by those skilled in the art within a scope of the technical idea of the invention.

For example, as in a 24th modification illustrated in FIG. 14, the treatment apparatus 10 may further include the detection unit 90 configured to detect fluorescence that is emitted by the antibody-photosensitive substance excited by being irradiated with near-infrared rays from the light-emitting unit 52 and has a wavelength (for example, 704 nm) different from a wavelength of irradiation light (for example, 689 nm). The detection unit 90 includes, for example, an optical waveguide 91 such as an optical fiber disposed in the irradiation lumen 25 like the irradiation unit 50 and receiving light, and an optical sensor 92 capable of detecting the amount of light. The detection unit 90 may include, at a position where the detection unit 90 receives light, a semiconductor sensor such as a CMOS image sensor that senses the light and converts the light into an electrical signal.

When the antibody-photosensitive substance bound to the tumor cells C is irradiated with the near-infrared rays, the antibody-photosensitive substance causes a photoreaction to emit the fluorescence, and destroys the tumor cells C. The antibody-photosensitive substance stops emitting the fluorescence after the tumor cells C are destroyed. Therefore, a degree of destruction of the tumor cells C due to the emission of the excitation light can be checked by measuring a change in an intensity of the detected fluorescence by the optical sensor 92. Therefore, a progress state of the photoreaction for destroying the tumor cells C can be checked.

The detection unit 90 may be a device different from the treatment apparatus 10 including the irradiation unit 50 described above as long as the detection unit 90 can detect the fluorescence emitted by the antibody-photosensitive substance excited by receiving the near-infrared rays. The detection unit 90 may be inserted into the vagina V, a uterus, a rectum, a bladder, a urethra, an abdominal cavity, a blood vessel, a ureter, or the like to detect fluorescence. The detection of the fluorescence by the detection unit 90 may be performed in parallel with the emission of the near-infrared rays by the treatment apparatus 10, or may be performed after the emission of the near-infrared rays by the treatment apparatus 10 is ended. The detection unit 90 may be inserted into the vagina V or the cervical canal CC after the treatment apparatus 10 is drawn out of the cervical canal CC and the vagina V. The detection unit 90 may detect fluorescence from a body surface outside a body in parallel with the emission of the near-infrared rays by the treatment apparatus 10 or after the emission of the near-infrared rays.

When the operator inserts the treatment apparatus 10 into the vagina V or the cervical canal CC, the detection unit 90 may be used to check a length of insertion of the treatment apparatus 10. For example, a position of the treatment apparatus 10 can be checked based on an image obtained from the CMOS image sensor or a change in the intensity or color of light obtained from the optical waveguide 91 such as an optical fiber.

The detailed description above describes embodiments of a catheter and operational method representing examples of the tumor cell treatment apparatus and tumor cell treatment method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A treatment apparatus configured to irradiate an antibody-photosensitive substance bound to a tumor cell with excitation light, the treatment apparatus comprising:
a main shaft including a lumen extending throughout the main shaft, the main shaft including a distal portion;
a distal structure portion disposed at the distal portion of the main shaft and movable together with the main shaft;
an irradiation shaft positioned in the lumen in the main shaft and axially movable relative to the main shaft, the irradiation shaft including an irradiation lumen;
the irradiation shaft including a distal portion at which is located a distal shaft, the irradiation lumen extending into a distal portion of the distal shaft;
the distal portion of the distal shaft being located distally beyond a distal-most part of the distal structure portion, the irradiation lumen extending through the distal structure portion;
the distal structure portion being configured so that an outermost surface of the distal structure portion is radially outwardly of an outer surface of the main shaft and radially outwardly of an outer surface of the distal portion of the distal shaft; and
an irradiation unit positioned in the irradiation lumen, axially movable in the irradiation lumen and configured to emit, from both the distal structure portion and the distal portion of the distal shaft that is located distally beyond the distal-most part of the distal structure portion the excitation light to irradiate the antibody-photosensitive substance.

2. The treatment apparatus according to claim 1, wherein the distal structure portion possesses a proximal end and a distal end, the distal structure portion including a through hole extending through the distal structure portion from the proximal end of the distal structure portion to the distal end of the distal structure portion, the irradiation shaft passing through the through hole and being axially movable in the through hole

3. The treatment apparatus according to claim 1, wherein a distal-most end of the distal shaft is closed so that a distal-most end of the irradiation lumen is closed.

4. The treatment apparatus according to claim 1, wherein the distal structure portion includes a connection portion that is connected to the main shaft, a diameter expanded portion that extends radially outward from the connection portion, and a wall portion that extends in a distal direction and axially overlaps an axially extending part of the distal shaft.

5. The treatment apparatus according to claim 1, wherein the distal structure portion is cup-shaped so that a radially outwardly located part of the distal structure is located more distal of a radially inwardly located part of the distal structure portion.

6. The treatment apparatus according to claim 1, further comprising: a detection unit configured to detect fluorescence emitted by the antibody-photosensitive substance when the antibody-photosensitive substance is irradiated by the excitation light.

7. A treatment apparatus configured to irradiate an antibody-photosensitive substance bound to a tumor cell with excitation light, the treatment apparatus comprising:
a main shaft including a distal portion and a proximal portion;
a distal structure portion disposed at the distal portion of the main shaft, the distal structure portion possessing an outer configuration that is larger in a radial direction of the main shaft than the main shaft in the radial direction of the main shaft;
a distal shaft protruding in a distal direction from the distal structure portion so that a distal end of the distal shaft protrudes distally beyond the distal structure portion; and
at least one irradiation unit configured to emit, from the distal shaft and the distal structure portion, the excitation light to irradiate the antibody-photosensitive substance.

8. The treatment apparatus according to claim 7, wherein
the distal shaft incudes an inside, the distal structure portion possessing a proximal end and a distal end, the distal structure portion including a through hole extending through the distal structure portion from the distal end to the proximal end, the treatment apparatus including an irradiation lumen communicating with the through hole and the inside of the distal shaft, and configured to movably accommodate the irradiation unit.

9. The treatment apparatus according to claim 7, wherein the distal structure portion is cup-shaped with a recessed portion formed on a distal side of the distal structure portion.

10. The treatment apparatus according to claim 9, wherein
the distal structure portion includes a wall portion surrounding the recessed portion and protruding in the distal direction, and
the wall portion includes, at a part in a peripheral direction surrounding the recessed portion, a protruding portion that protrudes further in the distal direction than all other parts of the distal structure portion.

11. The treatment apparatus according to claim 7, wherein
the distal shaft is configured to emit the excitation light in a direction substantially perpendicular to central axis of the distal shaft, and
the distal structure portion is configured to emit the excitation light in substantially the distal direction.

12. The treatment apparatus according to claim 7, further comprising: a detection unit configured to detect fluorescence emitted by the antibody-photosensitive substance when the antibody-photosensitive substance is irradiated by the excitation light.

13. The treatment apparatus according to claim 7, wherein the distal structure portion is configured to move relative to the main shaft in an axial direction of the main shaft.

14. A treatment method, comprising:
intravenously administering an antibody-photosensitive substance;
inserting a treatment apparatus into a living body 12 to 36 hours after the intravenous administration of the antibody-photosensitive substance, the treatment apparatus including a distal structure portion disposed on a distal portion of an elongated main shaft and a distal shaft protruding in a distal direction beyond the distal structure portion, the treatment apparatus being configured to emit excitation light of the antibody-photosensitive substance;
inserting the distal shaft into a body lumen;
inserting the distal structure portion into the living body;
emitting the excitation light from the distal shaft to a surrounding tissue to excite the antibody-photosensitive substance; and
emitting the excitation light from the distal structure portion to a surrounding tissue to excite the antibody-photosensitive substance.

15. The treatment method according to claim 14, wherein
the distal structure portion is cup-shaped, with a recessed portion formed on a distal portion of the distal structure portion, and
during the inserting of the distal structure portion into the living body, at least a part of the distal structure portion is inserted into a vaginal vault.

16. The treatment method according to claim 14, wherein
during the emitting of the excitation light from the distal shaft, an irradiation unit configured to emit the excitation light is disposed inside the distal shaft to emit the excitation light from the irradiation unit,
during the emitting of the excitation light from the distal structure portion, the irradiation unit is disposed inside the distal structure portion to emit the excitation light from the irradiation unit, and
the irradiation unit is moved between the distal shaft and the distal structure portion after the emitting of the excitation light from the distal shaft and before the emitting of the excitation light from the distal structure portion.

17. The treatment method according to claim 14, wherein the emitting of the excitation light from the distal shaft and the step of emitting the excitation light from the distal structure portion are performed simultaneously.

18. The treatment method according to claim 14, further comprising:
detecting fluorescence emitted by the antibody-photosensitive substance after the antibody-photosensitive substance is excited by the excitation light and checking an intensity of the fluorescence.

19. The treatment method according to claim 18, wherein the checking of the intensity of the fluorescence is performed in parallel with the emitting of the excitation light from the distal shaft and the emitting of the excitation light from the distal structure portion.

20. The treatment method according to claim 18, wherein the checking of the intensity of the fluorescence is performed after the emitting of the excitation light from the distal shaft and after the emitting of the excitation light from the distal structure portion.
